(19) United States
(12) Patent Application Publication
(10) Pub. No.: US 2024/0074662 A1
(43) Pub. Date: Mar. 7, 2024

(54) WEARABLE MONITOR CHARGER

(71) Applicant: Schalal Mudhir HABIB, Monaco (MC)

(72) Inventor: Schalal Mudhir HABIB, Monaco (MC)

(73) Assignee: KOS, Inc., PALO ALTO, CA (US)

(21) Appl. No.: 17/945,699

(22) Filed: Sep. 15, 2022

Related U.S. Application Data
(62) Division of application No. 17/903,740, filed on Sep. 6, 2022.

(51) Int. Cl. A61B 5/00; G16H 40/67

(52) U.S. Cl. CPC A61B 5/0022; G16H 40/67; G16H 50/20

(57) ABSTRACT

A charger to charge a wearable device while in use, the charger configured to transfer energy to the wearable device by one of wirelessly or direct connection through complementary pogo pads. The charger may have an LED strip to indicate power level; magnets to attach to the wrist band during charging; a plastic polycarbonate enclosure; a battery in the power pack having a capacity of >400 mAh. Where the charger uses a direct electrical connection using pogo pads, the pogo pads may be configured to be received by enlarged pogo pads on the wearable device whereby contact is maintained as a user moves and flexes the band or to adjust to the user's variation in wrist size. A power band magnet is placed close to the pogo pads and configured to be adjacent to a metal pad or complimentary magnet on a wearable device.

### FIELD

The present embodiments relate generally to wearable devices, methods and systems, and specifically to wearable devices, methods and systems configured to continuously monitor on board and optionally off-board sensors in communication with a monitoring and outputting application that uses machine learning to provide improving data analyses and recommendations derived therefrom.

### BACKGROUND

Wearable devices are known in the art, such as wellness and fitness trackers, smart watches, and the like. Such devices may have one or more sensors to collect predetermined data of a user from the top of their wrist. Such devices may have heart rate sensors (infrared & green LED+sensor), ambient light sensors, accelerometers for tracking movement, gyroscopes, microphones, touch screens, vibration generators, wireless connections to smart phones, GPS (global positioning systems), and the like. A user's heart rate may be determined by sensors that include infrared and visible-light LEDS in addition to photosensors, which all work together. The data may be used to simply monitor a person's daily activity. Such devices are typically marketed as wellness devices and are thus capable of analyzing data collected during limited periods of time and through limited number of sensors. Virtual examination and diagnosis through videoconferencing tools are also becoming popular.

Despite these advances in the art, there remains a desire and a need for advances and improvements in such devices, and methods, systems and applications for improving data analyses and recommendations derived therefrom.

Unless otherwise indicated herein, the materials described in this section are not prior art to the claims and are not admitted as being prior art by inclusion in this section.

### SUMMARY

Accordingly, to advance at least the aforementioned deficiencies in the art, described herein are continuously wearable devices, methods and systems configured to continuously monitor on board and optionally off-board sensors in communication with a monitoring and outputting application that uses machine learning to provide improving data analyses and recommendations derived therefrom.

According to one approach the present embodiments provide a wearable device configured for monitoring health of a user, including a wearable band comprising, one or more communicatively connected components attached to or embedded within the wearable band;
wherein the components comprise one or more biosensors; wherein the wearable band is sized to fit around a user's wrist, and when positioned around the user's wrist, the wearable band positions one or more biosensors at or near a pulse-taking location on the user's wrist.

The sensors in one embodiment may be included for heart rate, pulse oximeter, skin temperature, ECG, ambient temperature, accelerometer, gyroscope, a microphone, sensors to determine air quality, sound level/quality, light quality, and the like and combinations thereof.

The wearable device can be made from an elastomeric material capable of stretching to an increased circumference of at least 70% from its circumference at rest.

In another approach the present embodiments may include vibration/haptics, GPS, negative temperature coefficient (NTC) thermistors, NB-IoT/GPS, BLE Antenna Design, Li-Ion/Li—Po Battery. The wearable device can be configured to resist water penetration up to an immersion depth up to 15 meters for 30 minutes one or more of the components is encased in a plastic enclosure assembled using heat stacking/welding and/or adhesives. One or more components may be is encased in a plastic enclosure.

The present embodiments also include a charger to charge a wearable device while in use, the charger configured to transfer energy to the wearable device by one of wirelessly or direct connection through complementary pogo pads, the charger comprising an LED strip to indicate power level; magnets to attach to the wrist band during charging; a plastic polycarbonate enclosure; a battery in the power pack having a capacity of >400 mAh.

The charge may have a wireless charging mechanism and a Metallic (ferrous) Strip on its top side to hold it against a magnet in wireless charger. Alternatively, the charger may use a direct electrical connection using pogo pads, the pogo pads configured to be received by enlarged pogo pads on the wearable device whereby contact is maintained as a user moves and flexes the band or to adjust to the users variation in wrist size. A power band magnet may be placed close to the pogo pads and configured to be adjacent to a metal pad or complimentary magnet on the wearable device.

The present embodiments also include a method to monitor a wearable device, the method may have the steps of: providing a processing system located remotely from the wearable device having a memory that stores executable instructions that, when executed by the processing system, facilitate performance of operations, having: obtaining baseline biosensor samples of a user of the wearable device to establish expected biosensor outputs; obtaining continuous biosensor output samples at predetermined intervals; storing acquired baseline and continuous sensor outputs; comparing at predetermined intervals the continuous biosensor outputs to the baseline biosensor outputs; determining changes in the continuous biosensor outputs to the baseline biosensor outputs; and outputting information using machine learning associated with the changes in the continuous biosensor outputs to the baseline biosensor outputs.

According to one approach for this method, the step of outputting information using machine learning associated with the changes in the continuous biosensor outputs to the baseline biosensor outputs is used to establish a digital identity of the user.

In another approach, the step of outputting information using machine learning associated with the changes in the continuous biosensor outputs to the baseline biosensor outputs is by providing an interactive 3D chat bot (avatar) interface.

In another approach, the step of outputting information associated with the changes in the continuous biosensor outputs to the baseline biosensor outputs is providing the additional step of outputting direct feedback to at least one of a user or health care provider if an adverse health condition is indicated from the output of the one or more biosensors.

In another approach, the step of outputting information using machine learning associated with the changes in the continuous biosensor outputs to the baseline biosensor outputs may comprise providing a module configured to load data into a dataset;
providing a supervised machine learning module configured to initialize a predetermined labeled condition data set for human activities of daily living; acquiring user baseline sensor data values associated with at least one of a user demographic, activity level, health condition and environment from the labeled condition data set; providing a supervised machine learning module that once initialized accepts choice of label from the labeled condition data set for the user's current sensor values; and generating one or more supervised machine learning programs based on the labeled condition data set and user actual sensor data over time at predetermined intervals to continuously improve recognition accuracy of one or more user conditions and one or more human activities of daily living; wherein the supervised learning module comprise one or more of logic hardware and a non-transitory computer readable medium storing computer executable code.

Other features will become more apparent to persons having ordinary skill in the art to which the processes and methods pertain and from the following description and claims.

### BRIEF DESCRIPTION OF THE DRAWINGS

The present embodiments, as well as a non-limiting exemplary mode of use, further objectives and advantages thereof, will best be understood by reference to the following detailed description of an illustrative embodiment when read in conjunction with the accompanying drawings, wherein:

FIG. 1 illustrates an exemplary unassembled perspective wearable device shown as a wearable monitor/wrist band and power bank according to one embodiment.

FIG. 2 illustrates an assembled perspective wearable monitor and power bank of FIG. 1.

FIG. 3 illustrates an exemplary unassembled perspective view of the wrist band and circuitry.

FIG. 4 illustrates an exemplary assembled perspective wrist band and circuitry of FIG. 3.

FIG. 5a illustrates an exemplary exploded view of a power bank with pogo pad charging.

FIG. 5b illustrates and exemplary exploded view of a power bank with wireless charging.

FIG. 6 illustrates an exemplary perspective cutaway view of a power bank with wireless charging capability of FIG. 5b.

FIG. 7 illustrates an exemplary planar cutaway view of a power bank with wireless charging capability of FIG. 5b.

FIG. 8 illustrates an exemplary exploded bottom perspective view of the wrist band.

FIG. 9 illustrates an exemplary assembled bottom perspective view of the wrist band with the wristband material shown in hidden lines.

FIG. 10 illustrates a front view of the exemplary wrist band of FIG. 1.

FIG. 11 illustrates a back view of the exemplary wrist band of FIG. 1.

FIG. 12 illustrates a side view of the exemplary wrist band of FIG. 1.

FIG. 13 illustrates an exemplary legend of messages from the exemplary LED signal 164, shown as a capital K.

FIG. 14 illustrates a schematic for a system for use in implementing methods, techniques, devices, and applications of the present embodiments.

FIG. 15 illustrates an exemplary schematic representation of an application session with a user of the present embodiments.

FIG. 16 illustrates a functional decomposition of the present embodiments including the inputs and outputs.

FIG. 17 illustrates an exemplary flow diagram of the present embodiments showing activity monitoring

FIG. 18 illustrates an exemplary flow diagram of the present embodiments showing digital identification.

FIG. 19 illustrates an exemplary flow diagram of the present embodiments showing activity labeling.

FIG. 20 illustrates an exemplary flow diagram of the present embodiments showing health data acquisition and proposed diagnoses using machine learning.

FIG. 21 illustrates an exemplary flow diagram of the present embodiments showing a virtual doctor application.

FIG. 22 illustrates an exemplary circuitry configuration of the present embodiments for a wearable device.

FIG. 23 illustrates an exemplary circuitry configuration of the present embodiments for a wearable device.

FIG. 24 illustrates an exemplary circuitry configuration of the present embodiments for a wearable device.

FIG. 25 illustrates a system that may be used in processing signals in accordance with at least some embodiments.

FIG. 26 illustrates a system for use in implementing methods, techniques, devices, apparatuses, systems, modules, units and the like in providing user interactive virtual environments in accordance with some embodiments.

Corresponding reference characters may indicate corresponding components throughout the several views of the drawings. Skilled artisans will appreciate that elements in the figures are illustrated for simplicity and clarity and have not necessarily been drawn to scale. For example, the dimensions/peaks of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

While the features described herein may be susceptible to various modifications and alternative forms, specific embodiments thereof are shown by way of example in the drawings and are herein described in detail. It should be understood, however, that the drawings and detailed description thereto are not intended to be limiting to the particular form disclosed, but on the contrary, the intention is to cover all modifications, equivalents and alternatives falling within the spirit and scope of the subject matter as defined by the appended claims.

### DETAILED DESCRIPTION

Nearly 2,500 years ago on Kos Island in the southeastern Aegean Sea, Greek physician Hippocrates founded the science of medicine. By diagnosing disease through extensive research and observations, along with descriptions of symptoms to find an ailment, Hippocrates unknowingly became the first doctor in history. Since then, medicine has developed into an area of study that has one goal in mind: to save lives. Even today, doctors must swear to the Hippocratic Oath, which is a widely known Greek text describing the importance of upholding ethical medicinal practices.

Through the past two and a half centuries, medicine has changed with the times. In the Middle Ages, doctors would treat the Bubonic Plague as if it were a condemnation of religious torture. The Renaissance introduced the scientific method, which incorporated a whole new philosophy behind the practice itself. Now, medicine is influenced by technology. In Solow's Growth Model, which describes economic growth in the very long run, the advancement of human civilization takes place due to a single factor, and that is technological growth. The Solow growth model, also called the neoclassical growth model, was developed by Robert Solow and Trevor Swan in 1956. Robert Solow later received the Nobel Prize in Economics in 1987 for his work on this theory.

The Solow growth model is an extension of the Harrod-Domar Model. It states that there are three factors: technology, capital accumulation and labour force that drive economic growth. (See generally, https://www.romeconomics.com/beginners-guide-solow-growth-model/)

According to the present embodiments disclosed herein, the amalgamation of medicine and technology are at the forefront of modern health care.

The present embodiments are an application of investigating the symptoms of various diseases to build databases for predetermined diseases. These databases came from literature research to build a model for said predetermined diseases. With extensive data points, the models can be used to take predictive measures to catch disease in early stages.

In taking the Hippocratic oath, doctors swear to share scientific findings with the medical community for the betterment of society as a whole. From Greek medicine to modern surgery, the importance of bettering the practice has allowed for the improvement of medicine.

Accordingly, the present embodiments provide a versatile, smart tracking band (aka, wearable, wearable device, wrist band, and the like) that enables a comprehensive and intelligent health monitoring experience. The present embodiments leverage cutting-edge sensors, efficient IoT network technology, and artificial intelligence to ensure secure and rapid data transmission and interpretation. This enables supervisors of health ecosystems—like hospitals and elderly homes—to immediately react to unexpected incidents detected by an automatic diagnosis engine.

The present embodiments work together as a single network seamlessly connecting physician and patients together to give them the tools they need to focus on delivering frontline medical care more efficiently.

The present embodiments utilize new BioSensory concepts and engineering to connect applied science in a wide range of medical applications through wearable technology. This allows for blended biology and technology to develop a new generation of BioMetrics and BioFeedback that will serve to improve user well being through virtual ward monitoring.

With AI components the present embodiments will be part of a new wave of wearable technologies with augmented reality with greater capabilities to handle enterprise data. Taking an enterprise approach to managing patient care allows the present embodiments to bring the hospital home particularly for the most vulnerable.

Accordingly, the wearable of the present embodiments is an IoT enabled healthcare wristband designed to collect vital parameters of the wearer. According to one approach, the wearable may have the following built-in on-board sensors: heart rate; pulse oximeter; skin temperature; ECG; ambient temperature; accelerometers; gyroscopes, vibration, GPS, and the like including offboard sensors (e.g., cameras, blood pressure cuffs, and the like and combinations thereof). The present wearable is enabled to transmit the vital parameters of the wearer at regular intervals to Cloud using connectivity (BLE, NB-IoT). NB-IoT referred to as Narrowband-IoT is a narrowband radio technology. BLE refers to Bluetooth low energy protocol stack (or protocol stack).

A user/wearer may track their health information using a Web/Mobile application included in the present embodiments. The application designed to understand and differentiate Activities of Daily Living (ADL) activities. The artificial intelligence/machine learning (AI/ML) model developed for the application assists in capturing, recognizing and classifying images using camera and diagnose and/or assists in diagnosing the symptoms using the Avatar chatbot.

Artificial intelligence algorithms are designed to recognize and classify specific configurations of data. Machine learning is to recognize the data patterns and to improve AI algorithm performance through training the algorithm with training datasets.

The user interface of the present embodiments may have voice based conversational AI chatbot acting as a virtual doctor/Digital Avatar The bot is designed to track and monitor health information of the user. It can assist the wearer in diagnose the diseases, in staying healthy, scheduling appointments, notifying medications, and other various calendaring activities.

The bot can self-learn using ML/AI from Medical Knowledge bases of the present embodiments, compare the symptoms of the wearer, diagnose symptoms to identify diseases and suggests corrective steps. The bot can assist in identifying diseases like, for exemplary purposes, Fibromyalgia, Cold-Flu, Adenoviruses and Skin-cancer, identifying Mental Health problems, and the like.

The user's mobile application enables users to interact with a Virtual Doctor that will help to identify disease. It can also enable access such features as Device Management, Mobile to Device communication, Mobile to Cloud communication, Patient Appointment workflow, and the like.

The member web application enables doctor to interact with a Virtual Doctor that will help to access the features—Doctor Registration, Doctor Appointment, Patient Diagnose, and the like.

In one approach, no personal Data is used for registration and Login. For example, the system may recognize the user based on the sensor data that is used as a user ‘fingerprint’.

With recent advancements in Patient Monitoring and Bio Innovation, the present embodiments allow possibility to diagnose diseases in their earliest stages and bring back ethical governance to the user. Our advanced platform and wearable provided herein can detect a condition with an improved accuracy beyond that known in the art, hopefully even before serious progression, even if the disease is unknown by the user.

By way of example, we consider the case study of the Zika Virus between 2015 and 2017 with early detection. The Zika virus developed swiftly into an epidemic of global proportions. Discovered approximately 70 years prior, Zika remained primarily unrecognized until 2007 and 2013 when the Pacific region first reported outbreaks. The statistics for the Zika virus are staggering and its rampant increase bewildering for Doctors and patients. A whopping 70 to 80 percent of Zika Virus infections show no noticeable symptoms, meaning that nearly 4 of 5 individuals infected with the disease are unaware they were sick. In just 20 to 30 percent of cases, the condition presented only mild symptoms within 3 to 12 days. Consequently, Zika can spread rapidly and new Bioreporting tools to understand data changes in its movements and mutations is desirable. On the rare occasions that symptoms were noticeable, they were often confused for other viral infections, such as adenovirus, dengue, chikungunya, or West Nile Fever. Like malaria, typical physiological symptoms of the Zika virus involved a mix of low-grade fever, limb and muscle pain, and headache.

The long-term implications of this disease are heartbreaking particularly when patient monitoring cannot reveal BioData back to physicians. Had the technology existed to detect the Zika virus in its earliest stage during this time, intervention and treatment would have saved many lives. With the present wearable device and advanced telemedicine software, life-saving disease detection is now a potential reality with secondary opportunities for disaster relief beyond virus outbreaks.

Technological advancements should prioritize human life. By way of artificial intelligence, health care professionals, the present embodiment's platform and wearable device, it is possible to detect minor fluctuations user biodata that could indicate any underlying illnesses whether it's cancer, heart disease or viral infection. The present embodiments use ultra-sensitive sensors to continuously monitor user data, which is stored permanently, compared with the data collected, algorithms using ML/AI work to detect a health condition sooner than known in the art. This early detection allows for better patient outcomes.

By way of another example of the usefulness of the present embodiments, the medical discipline of mental health includes our emotional, psychological, and social well-being. It affects how we think, feel, and act. It also helps determine how we handle stress, relate to others, and make choices. Mental health is important at every stage of life, from childhood and adolescence through adulthood.

Over the course of your life, if you experience mental health problems, your thinking, mood, and behavior could be affected. Thus, many factors contribute to mental health problems, including: biological factors, such as genes or brain chemistry; life experiences, such as trauma or abuse; family history of mental health problems; and the like. Mental health problems are common but help is available. People with mental health problems can get better and many recover completely.

Depression is more than just feeling down or having a bad day. When a sad mood lasts for a long time and interferes with normal, everyday functioning. Exemplary symptoms of depression include: feeling sad or anxious often or all the time; not wanting to do activities that used to be fun; feeling irritable, easily frustrated, or restless; having trouble falling asleep or staying asleep; waking up too early or sleeping too much; eating more or less than usual or having no appetite; feeling tired even after sleeping well; feeling guilty, worthless, or helpless; and the like.

The present embodiments can collect, evaluate, analyze, and interpret available medical and biotechnological information. The involvement of medical artificial intelligence in healthcare can assist to assess and anticipate health risks. This form of artificial intelligence is expected to play a increasing role in healthcare because of the advances in learning algorithms, computing power, and the large data sets that are available, which can be sourced from medical records. The computing power of the present embodiment's intelligent software increases rapidly due to the availability of wide graphics processor units, which makes processing even faster. Big data is supported as well by the endless storage in the cloud.

Learning algorithms are becoming more accurate and precise for the present embodiments as they interact with large training data sets catering for the development new insights into diagnostics. The rise of medical intelligence in this era of massive data helps physicians improve the quality of patient care. The medical intelligence of the present embodiments is well suited to manage large data sets, handle repetitive work, and provide improved judgment support that reduces errors. Accordingly, the present medical intelligence will have a significant impact on healthcare, such as clinical decision making and chronic disease management.

Despite being at the early stages of adoption, medical intelligence is showing great promise in areas such as pathology, radiology, cardiology, and ophthalmology. Medical intelligence can make physicians such as radiologists more effective in serving their purpose. Learning for the present embodiments is from experience, AI and ML and not from being explicitly programmed. This is in contrast to traditional computer programs which are require to be instructed in a step by step method. The system learns to process and detect from the data provided to it during training sessions.

Medical intelligence can provide fast and accurate diagnostics. Many diseases require immediate medical action because they can become more severe rapidly. For an artificial neural network, it is much easier to diagnose medical conditions earlier because of their ability to learn from previous cases.

The present embodiments can be a personal digital doctor on your wrist proving: eSIM (a digital SIM that allows you to activate a cellular plan from your carrier without having to use a physical nano-SIM) with global coverage; self powering; elegant & lightweight. In short the present embodiments give people a simple way to retrieve health advice and connect to caretakers with a unique machine learning health platform. The present wearable device is a continuously (e.g., permanent) wrist-worn health assistant allowing you to access integrated health services. The wearable, for example, collects vital information like heart rate and skin temperature and provides a single channel ECG measurement connected to a mighty health platform. Data collection and sensor fusion can be based on machine learning algorithms and takes place at different times during a day for deeper insights and more personalized health advisories. Some exemplary capable features from the present embodiments are: the wearable, the power bank, early disease detection; application of vital and activity data to recognize indication of diseases in early stages; emergency detection; wearable will automatically send alarms to predetermined emergency contacts and caretaker in near real time; a medical assistant; learned reminders for medication and supplements routines are monitored by your personal health assistant reminding you to take or recorder medication on time; and the like.

As for personal health, the personal health assistant of the present embodiments learns from a user's daily activities and supports the user with health advisory and automated tasks related to the user's health treatment.

A health calendar is used to watch all health and medical-related appointments in the user's personal health calendar. Further, all relevant information are presented in your personal health dashboard.

In the art, patients and medics suffer under current health care practices. Avoidable visits block medical capacities. Increasing waiting periods at the doctor. Increased waiting periods to get appointments. Repeating procurement of medicines causes long term efforts for patients. Amount of personal medical data increases with every visit at different doctors. Depending on area, no access to medical treatment and advisory. Depending on area, no access to medical treatment and advisory. As to caretakers, medical personnel are struggling to obtain treatment quality for the increasing demands. Avoidable visits bind medical capacities significantly and affect the treatment quality of all patients negatively. Less treatment time for each patients causes stress within medical providers and patients.

Accordingly, the present embodiments provide AI remote based bio diagnostics through machine learning. The present AI Platform is created to empower both the patient and the front-line health care worker so that both can understand and engage with each other reducing information gap risk factors to bring a range of BioSensory information to all parties. Such scores allow closer mediation in time-sensitive situations and allows users/patients to highlight patient sensitive issues with early recognition to understand prognosis data indicators earlier.

The present embodiments may create a digital physician environment. The desire for Self Managed Health is rising. The present embodiments provide an augmented reality tool to provide better patient care by providing better tools engaging in patient care as well as better transparency on disease prevention through better health education. COVID-19 has highlighted not only the heightened burden on public services, but also the need for better information and clearer data analysis to monitor and analyze preventative changes.

The present embodiments help optimize health care with personalized wearable BioSensory technology to streamline frontline medical services by reducing patient waiting and hospital ward waiting times through creating immersive telemedical channels through better data connections. Care continuity is a priority in registering patient BioFeedback to help reduce information risk and to highlight vulnerable patients who show earlier symptoms first reducing stress for the care system. The present embodiments provide medical grade notifications to enhance patient security and to provide accurate monitoring for clinicians. Tracking symptoms and anticipating treatment through monitoring vital signs is key to creating a more enhanced patient care plan.

Biofeedback is an important aspect in making the data platform the key advisory platform in preventative medicine. Digital Therapeutics allow users to understand their baseline scores and create further advances in their well being. Digital reorganization requires big computing, balanced with patient data confidentiality with their main physician by making sure that the user can control how their information is relayed and understood by different parties. In some arenas this can mean picking up medical errors and cleaning with better datasets; in others it can mean looking through pools of datasets and understanding where the trends are for correct patient spending.

BioScience is the heart of the present AI platform offering an extensive learning resource. From myth busting and must-know basics to managing specific symptoms, the present embodiments can output what is needed to know with character and care.

As for BioMarking, as the present artificial intelligence platform evolves, data modes can be connected in randomized experimental stress tests to understand where the critical junction points occur in patient wellbeing. This not only provides better insight to patient health trends but allows for better education empowering well being and understanding of future potential risks. With benchmarking the present algorithms can then assess individual health risks rather than taking a summary of data trends for better informed advice using the platforms clinical measurements than presently known in the art. Deep data requires understanding the individual patient at a deep level so that detecting clues that predict disease can be understood more clearly at an aggregate level where data noise can be cleaned out earlier preventing errors being determined in Biofeedback

As for BioTherapeutics the present platform can lead to better self-assessment such as the case within physiotherapy whereby self-referral eases stress on frontline healthcare services. There can also be voluntary health question sets which can help provide a basis for further patient education and better patient examination.

As for BioMetrics, in the realm of digital assessment, remote patient monitoring doesn't just need a sensory but an interactive environment. Patients are more willing and can be better understood through better sensory engagement. Digital biomarking with the present embodiments works through a controlled app environment requiring the user to engage with the AI platform which will assess, give feedback creating better engagement between the patient and their physician.

As mentioned, the present embodiments drive health homeward, while improving personal health and wellness at a lower cost and higher quality. It's about all of us making the best choices and decisions possible as we strive to enrich our lives and the lives of those around us while simultaneously taking in account the health and wellness aspects of those choices. To do this it is critical that we have timely and accurate information about our health and the options we have.

The information age brought with it tremendous access and, at the same time, the ability to respond to opportunities and risks a record speeds. The result: we have the opportunity to monitor healthcare quality, its actual impact on our health, and eliminate excessive cost, i.e., costs and health impact can be aligned—we can monitor and assess the impact of our health and wellness choices in real-time to determine the health and wellness value of our decisions! To respond to this opportunity, the present embodiments provide a health care platform that powers and creates health and wellness value-based programs.

Leveraging advanced data analytics, technology, and also nationwide healthcare provider networks, the current platform helps ensure the success of healthcare consumers, payors and providers through their participating in value-based care programs.

By activating the home as a key part of the care continuum, we lessen dependence on facility-centric care, prevent costly adverse events, and facilitate holistic condition management across settings of care.

The present embodiments align financial incentives around health outcomes. By engaging people where they are daily, we help them stay healthy and independent at home and support their recovery homeward as part of the episode of care. This helps our customers reduce costs, improve the quality of care, and foster better experiences for individuals, all while reducing financial risks and expanding opportunities to impactfully participate in health and wellness value-based programs.

The present embodiments approach the modern challenges of health and wellness, including quality of care and cost, by leveraging information technology, smart devices, and the Internet of Things (IoT) to bring health and wellness practice into the home and our daily activities. The present systems observes and monitors a user's health continuously and informs decisions and the care of healthcare professionals. It contextualizes this information to help users and their our care givers to better understand how health and disease express and are experienced in our daily lives.

The present embodiments provide health and wellness outcomes in a multidimensional way, including dimensions of patient experience, treatment strategy and treatment efficiencies. These dimensions can be viewed as a multidimensional array of treatment goals, states and actions, this array can be displayed or represented as a geometric cube. Patient activity, symptom monitoring and analytics are integrated. Through the combination of wearable monitoring technology and AI/ML-enabled analysis in the context of daily activities, the present embodiments enable users to optimize outcomes against treatment and treatment costs.

Doctors, care givers, payors and patients are brought into closer collaboration through the present systems. Continuous monitoring of patient condition and analytics that learn how disease expresses itself in each individual can assist the physician in providing personalized and more effective and efficient treatment, enabling the treating physician to assess impact and patient compliance with treatment strategy to adjust treatment in real-time, including scheduling timely follow-up and adjusting treatment strategy.

Together with the expanding telemedicine, and enhanced engagement with and input from patient, the present doctor-patient ecosystem is more integrated, and doctors are able to respond more rapidly to evolving patient condition and treatment opportunities.

Laboratories, either those engaged in medical or pharmaceutical or medical devices R&D or those providing laboratory services to doctors and clinics, can also benefit from the present embodiments.

The present technologies, and its AI/ML-based data analytics, can assist in: treatment impact and efficiency; enhancing patient experience; illuminating treatment cost structures; and testing and validation of treatments, treatment strategies and technologies;

Pharmacies, and the treatment components they provide to the patient, are a critical element of the overall treatment strategy. Their timely awareness of the doctor-proscribed pharmaceuticals, including characteristics and cost, is critical to treatment impact and success.

The present embodiments suite not only provides the patient-authorized physician access to the patient's monitoring data and data analytics, but also assists the patient in scheduling and tracking appointments and allows the patient's physician to enter scripts/prescriptions directly into the physician interface of the present system.

In addition, using the present mobile or Web application: the patient is able to configure his/her pharmacy preferences; the doctor scripts/prescriptions can be directly passed on to the patient-configured pharmacy electronically and securely, and the pharmacy can notify the patient, and the prescribing doctor if desired, of the current status of the script, and the pharmacy, or pharmacy delivery partner, can arrange for the delivery of the prescribed medications to the patient and even confirm delivery.

Disease variants, their severity and frequency, even how the diseases express themselves in different population groups, are often difficult medical and statistical challenges. The disease data our institutions currently have is too small a dataset to draw statistically significant conclusions. The present embodiments enable continuous learning over time and so results in a more and more reliable classification of symptom patterns, both in an individual patient and across patient populations. Then in-service learning and ML algorithms allow the present system to optimize and so guarantee continuously improving classification. Disease characteristics and evolution, including disease variants and their severity and frequency, can be better understood for the individual patient and across patient populations.

Most governments around the globe have agencies that conduct research related to disease and health and regulate the pharmaceutical and medical devices used to treat patients. The present embodiments can be a resource to these government agencies in performing these functions by providing insights into characteristics and evolution of disease, and insights into how diseases express themselves in individual patients and across patient populations.

The present embodiments culminate in a 3D digital avatar for voice chatbot; a wrist band wearable and an external charger for charging the wrist band. AI/ML model development provides data Ingestion from wrist band into the database. Some points of novelty include NBIoT (narrow band-IoT) Antenna Design; battery design; derivation of a unique digital ‘signature’ of each user, and labeling the ADL activities and diseases in an automated way by the machine learning model.

The interaction between the user and the application happens through a 3D digital avatar acting as a human chatbot. During the conversation with the user, the Mobile application uses a healthcare symptom checker to detect any symptoms related to known diseases and symptoms. The application also has custom conversations to understand the health issues of a user and accordingly schedule the appointments with a doctor.

The present IoT (Internet of things) platform creates means for common language between patients and health professionals, eliminating information gaps. IoT platform outputs data which can be used to improve public healthcare domain. Patient profile is built on unique sensor footprint, not on user personal data. For accurate data labeling and self-learning, AI has an uninterrupted access to a complete and continuous (24/7) data captured through device sensors and human-machine interactions; and open source medical databases. AI can provide valuable health insights on its own and act as an intermediary interface between the patient and health professional. AI can provide real-time psychological assistance. Reduced medical cost to the end-user and reduced insurer cost can be realized.

The present embodiments can be a medical product and serves the recording of certain body-related measurement data for the later evaluation and processing of this data by further algorithms and data analysis to build a digital service. The wrist band serves as a data collector and gateway and provides the corresponding data to enable the digital service behind it. The digital service can be understood as a digital doctor, who communicates with the user via a web interface and on the basis of the measurement data, etc. and can make medical recommendations. In addition to medical (preventive) recommendations, the digital doctor should also recommend medications and provide links such as to refer online pharmacies where the proposed drug can be purchased directly by the user. In one approach the appearance of the digital doctor changes over time or may be individually adapted to the user on the basis of certain data.

After receiving a present device, the user should log in to the web interface and enter predetermined body-relevant data manually (e.g. body weight, height, gender and the like). During this process, the integrated SIM card should be activated by the end user. Another function is the input of personal emergency contacts that are contacted by the system in an emergency. Furthermore, the user should ideally talk to the digital doctor once a day. The results and forecasts created on the basis of this measurement data should be communicated to the user and questions should also be asked by the digital doctor, whose answers can be included in the health assessment of the user. The interaction between the user and the digital doctor takes place, among other things, via manual input of the user (input of the body-relevant data) and questions, which are placed by the digital doctor to the user.

The present device should be created once according to specifications and then no longer must be removed. The idea is that the device remains continuously/permanently attached to the user's arm as a permanent companion and protector. This requirement requires a sophisticated battery management and charging system for the product. According to one approach, the wrist band may have two batteries, one of which supplies the system until it is discharged. When the battery is discharged, the bracelet is further supplied by the second battery (there is no energy idle), while the first battery is removed and can be connected to a charger. One way to increase the battery life is through various energy harvesting measures (e.g., energy conversion by movement or by existing electromagnetic fields). Preferably, a charging bank is applied to the wrist band.

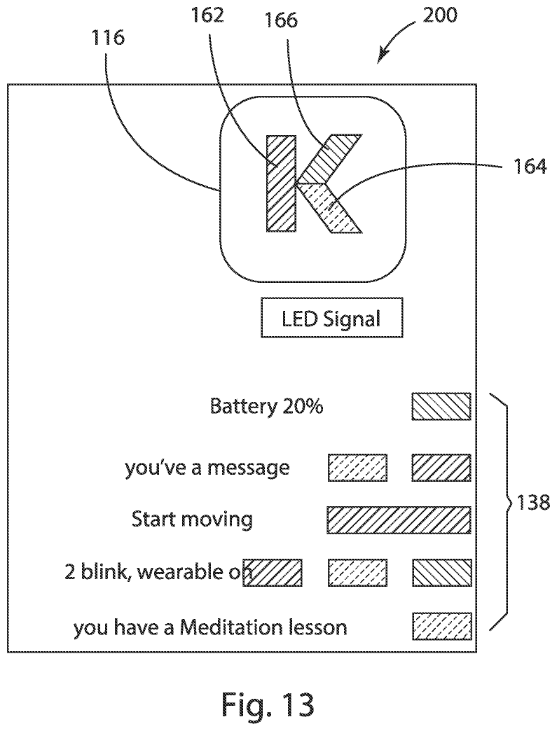

As a brief discussion of exemplary product features, the entire sensor system can be located at the bottom of the wrist. A variant wristband for men and a variant for women is envisaged. The display of the current embodiments is discreet (not eye-catching) if it is switched off. No color display is required (matte display). In one approach the display could show only two values (heartbeat and body temperature), or preferably none at all. LEDs may be used to signal different device states. According to the approach shown in FIG. 13, three LEDs are shown. The states can be defined as Status connected, status message available, battery status.

Material specification can be oriented to existing successful solutions to have a high load capacity. The operating instructions should be made available in digital form.

The device should recognize the user based on the measured data. A device should therefore only be used for one person at a time and should not be transferable. With the exception of a firmware update and SIM card activation, the user should not have to make any settings on the device to use the device.

The device can optionally be equipped with GPS to transmit the position dates in an emergency. It is noted that this option may be switchable to protect battery life.

Data transmission from the device is encrypted due to the sensitive application. The device should have a water resistance of 5-10 m and be functional at a height of up to 1000 meters. Optional features may include a mini-USB cable for both the device and/or the charging bank; a mobile APP where a user can log in to the web portal via their browser in their smartphone and use the services. The digital doctor must be fed with predetermined medical information before the system is launched. An ECG measurement may also be included as a one, two or three point/channel measurement. The index finger in one embodiment can be placed on a correspondingly sensitive surface of the wearable device. One approach can utilize the 3-point ECG measurement to use two measuring rings removable from the device, one of which is around the other (free) wrist and the others are placed around the ankle. This would allow a 3-point ECG measurement. Preferably a single channel ECG is utilized.

The present embodiments provide charging sources to power a built-in battery for wearable devices, such as wearable monitoring devices, and specifically to a removable direct transmission or wireless battery charging source that facilitates continuous use of the wearable device and machine learning to optimize use.

According to one embodiment, the one or more batteries may be configured to allow continuous wear and usage of the wearable system. In one embodiment, the wearable system may include two or more batteries. The system may include a removable battery that may be recharged using a charger. Additionally, the wearable device may include a chargeable built-in battery that can be recharged by a removable battery that can piggy-back onto the wearable device while still worn by the user. Thus, when the built-in battery is being charged, the user does not need to remove the wearable and may continue collecting data using the built-in battery. In other embodiments, the two batteries may both be removable and rechargeable.

In some embodiments, the removable charging battery and the wearable device may allow for wireless charging. In other embodiments, the battery may be a rechargeable via motion. In yet other embodiments, the battery may be rechargeable using a solar energy source or other energy capturing means.

The wearable device described herein may additionally be referred to in many exemplary terms such as an IoT (Internet of Things) Device, a device, a wearable device, a wearable, a wrist band, and the like. All these terms refer to device 122 as shown in the figures. The device is a smart wearable device enriched with multiple biosensors used for measurement of Vital signs (like Heart rate, ECG, Body Temperature), health, environment, activity of a user and the like. In one approach, wearable 122 collects the data and upload the data over the cloud through mobile using BLE 5 connectivity. Also, wearable 122 may transmit data over cloud-based server using NB-IoT connectivity in absence of BLE and mobile network unavailability. The user can visualize the data available through web user application. An accelerometer and gyroscope sensor can be used for fall detection and GPS module to send the coordinates over the BLE/NB-IoT in case of alarm. As discussed herein, wearable 122 can be charged by a power bank 120 through Pogo pins and may have RED, GREEN and BLUE color LEDs for status indication (See e.g., FIG. 13). In another approach, sensor/components may include NB-IoT Module; BLE Module; Pulse Oximeter/Heart Rate Sensor; Inertial Measurement Unit (IMU) Sensor; Single channel ECG; Digital Temperature sensor; Ambient Temperature sensor.

The band of wearable 122 may be a combination of materials to accommodate the sealing, stretching and strength. In one embodiment the an elastomeric polymer may be used to that can be stretched to more than 70 percent of its circumference at rest. The electronics and sensor assembly to be placed in sealed enclosure 154, such as a transparent plastic enclosure. The plastic enclosure may be further be covered by transparent rubber material by over molding process.

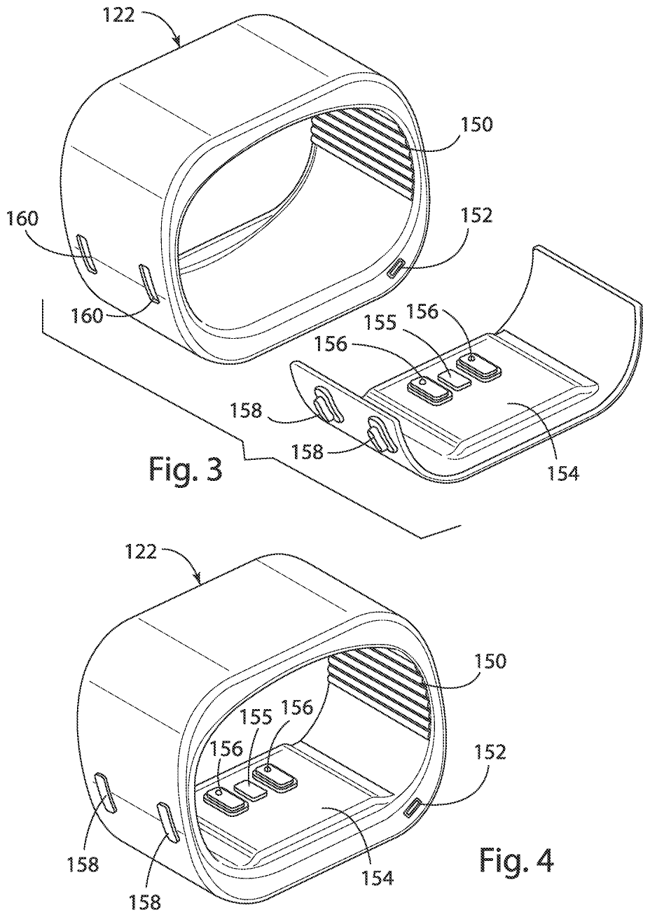

In one approach wearable 122 may be a transparent material to be used. The plastic material may be a poly-Carbonate. The rubber material may be silicone rubber. Material for ECG Electrodes may be Stainless Steel—SS 304. The material for Pogo Pads may be brass with nickel chrome plating. As for color, the wearable 122 can be white or translucent silicone rubber and the plastic enclosure 154 being clear. The appearance of the rubber band can be transparent in looks. Anti-slip features and air ventilation features 150 can be added on the inner side of band 122. (See e.g., FIGS. 3 and 4) In one approach, wearable 122 has an internal Battery; NB-IoT Antenna; BLE Antenna and sensors for NBIoT Module; Pulse Oximeter/Heart Rate Sensor; IM U Sensor; Single channel ECG; Digital Temperature sensor; and Ambient Temperature sensor.

In another approach, the sensor data may include data inputs from two sources, including sensor data inputs from wearable 122 and data inputs from the user while logged into the Web-based application, including Heart rate; Pulse Oximeter; Skin Temperature; ECG; Ambient Temperature; Accelerometer; Gyroscope; Vibration and GPS.

As for components in the plastic enclosure 154, sensor placement can include negative temperature coefficient (NTC) thermistors; NB-IoT/GPS and BLE Antenna Design can use known antenna for NB-IoT/GPS as per size shape available in the device. Known antenna for BLE can be as per size and space available in the device. The battery may be Li-Ion/Li—Po Battery having a capacity of 185 mAh. Device 122 can be IP-68 rated with following conditions for water ingress: water Immersion depth up to 15 meters; time duration for Immersion is 30 minutes. Enclosure 154 may plastic enclosures to be assembled by an adhesive. In another approach, enclosure 154 can be assembled using heat stacking/welding.

Wrist band is preferably of equal thickness on the top and bottom

Data from the device can be transmitted to a cloud host. Data acquired from a user's wristband can be transmitted in batches from the wristband, over a local wireless network, to one or more Cloud servers that host the present Web-based application and chat bot. Data acquired from a user's wristband is stored in a Sensor Data Storage database on the Cloud servers.

While logged into the Health Information Application the user can: input data; request reports and histories; input responses to questions posed to the user by the chat bot; choose a ‘label’ from a selection of Activities of Daily Life (ADL) such as walking; cycling; running; idle; sleeping; and many others.

A master medical knowledge database can access medical knowledge such as illness names and symptoms from known sources such as the Centers for Disease Control and Prevention, including the sensor data monitored and transmitted by the wearer wristband.

Digital Identity Management can include association of sensor data from sensor data storage and can be managed by a Digital Identity Management (Digital ID Mgt.) algorithm. The Digital Identity Management component can: create digital IDs that are different from the login credentials of the User; associate sensor data batches acquired from the wristband of the wristband wearer with the user's digital ID. The system can train the AI algorithms of data batches and the ADL label provided by the user using supervised machine learning. The user can access and enters data in the User Activity Health Information Database. Differential diagnoses can then be generated to the Health Information Application. The User Activity Health Information Database can be accessed and queried by the Digital Identity Management component and contains records that include: the User's digital ID; ADL labels and user's sensor data associated with the ADL labels,

The Diagnosis Engine can query the Master Medical Knowledge Data, reasons about medical data with user sensor data, received user sensor data with requests for a Differential Diagnosis from the Digital ID Management component and provides this differential diagnosis to Digital ID Management.

The present embodiments provide a means to charge the wearable monitoring device (wrist band) while still worn by the user. This power bank allows uninterrupted/continuous monitoring from the wearable device. The power bank body of the power bank can be configured to ‘hug’ or ‘piggy-back’ onto a portion of the circumference of the wearable monitor and constructed of the same materials, textures, compostions, and the like of the wearable device. The power bank can be attached to the with Wristband with the help of magnet placed within the power bank housing and be electrically connected to the wristband by pogo pins to charge the wristband battery or wirelessly using circuitry known in the art.

Several features of a power band are possible. In one embodiment, the power bank may have dimensions of a length of 6.23±0.1 cm; a width of 4.5±0.1 cm and a height of 5.7±0.1 cm. The power bank may have a micro-USB or other wired connection for charging in the wrist band as a secondary charge in option. The power bank may have a switch button for On/Status Check function. An LED strip can be provided to show the status of the battery charge. As shown, the LED strip has an exemplary four LEDs. The power bank can attach to the wrist band via a Magnet, which can be used to hold it against the wristband during wristband battery charging. The power band may be formed as plastic enclosure such as a polycarbonate, and even as a transparent polycarbonate. Optionally, the powerband may have ingress protection or water resistance properties. The battery in the power pack may have a capacity of >400 mAh. The plastic enclosure of the power bank may be held together by adhesive or Heat Stacking/welding to be used.

Metallic (Ferrous) Strip on Power Bank to on its Top Side to Hold it Against Magnet in Wireless Charger.

Another approach uses a direct electrical connection. In use in this embodiment, the power bank is attached to the wristband by enlarged pogo pads. The enlarged pogo pads allow the power band to maintain contact as the user moves and flexes the band. The connection between the pogo pads and the pogo pins may also slide to adjust to the users variation in wrist size. The power band magnet is placed close to the pogo pins to increase the magnetic connection between the pogo pads and the pogo pins.

Optionally, the power bank may feature wireless charging of the wrist band by placing various combinations of metal strips and magnetic strips and adjacent metal or magnetic strips in the wrist band so that the power band and wrist band connected by an Rx charging coil.

The present embodiments system includes a hardware wearable device that gathers patient medical data and transmits that data to a computer-based machine learning software application. The system may also include a mobile software application that can be installed on a smartphone or similar mobile device and allows the patient to access their medical data and the medical assistant software. The present system also includes a master medical database, hosted on a server with the medical assistant software or on a server networked with a server hosting the patient's medical data and the medical assistant software. The master medical database contains nominal medical data associated with a variety of patient conditions, continuously learns and records patient-specific medical data associated with a patient condition in real time using supervised machine learning. The system also includes a computer-based application, referred to as the virtual medical assistant that provides access to a patient's medical data, interviews the patient through a chatbot that represents graphically and in speech the medical assistant, authenticates the patient by means of a digital identity comprising, for example, a sequence of patient uploaded images (three or more), and analyzes patient data, together with patient-medical assistant interview responses, to produce a differential diagnosis together with suggests actions that may be taken by the patient as to address each of the possible patient conditions reported to the patient in the differential diagnosis.

In summary, the present system has several software and hardware components. A wearable hardware device is worn on the wrist of a patient that contains a sensor array for gathering patient medical data, an onboard rechargeable battery, Wi-Fi and Bluetooth antenna and communications software for transmitting patient medical data over a network to a computer that hosts the computer-based application, and a battery charging pack that can be attached to the wearable device to charge the onboard battery. A hardware battery charging pack that can be charged itself when, for example, connected to an electrical outlet using a mini-USB port and can be attached to the wearable device to charge the onboard rechargeable battery in the wearable device. A mobile application can be installed on a smartphone and can be used to access the patient medical data and the medical assistant software hosted on a computer host or server. A computer-based application can be installed on a desktop or laptop computer and used to access the virtual medical assistant, including the patient's medical data and the medical assistant software on a computer host or server.

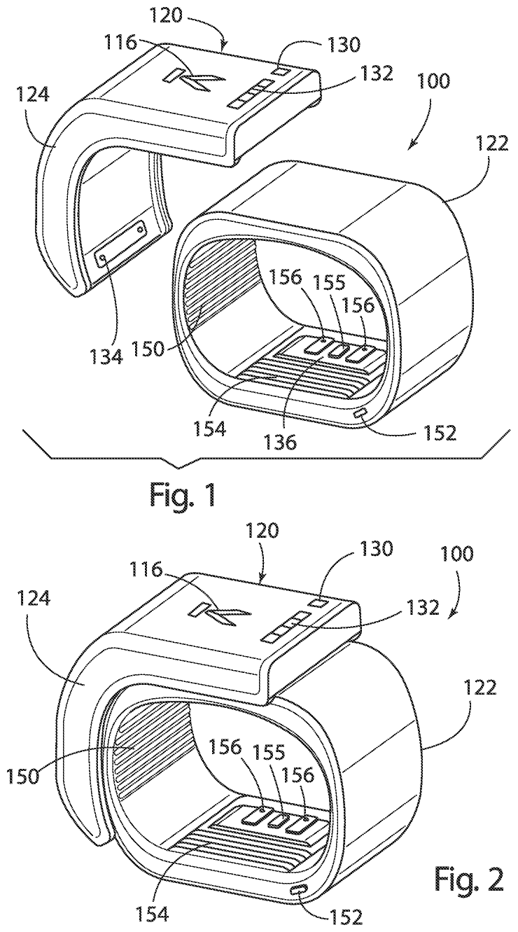

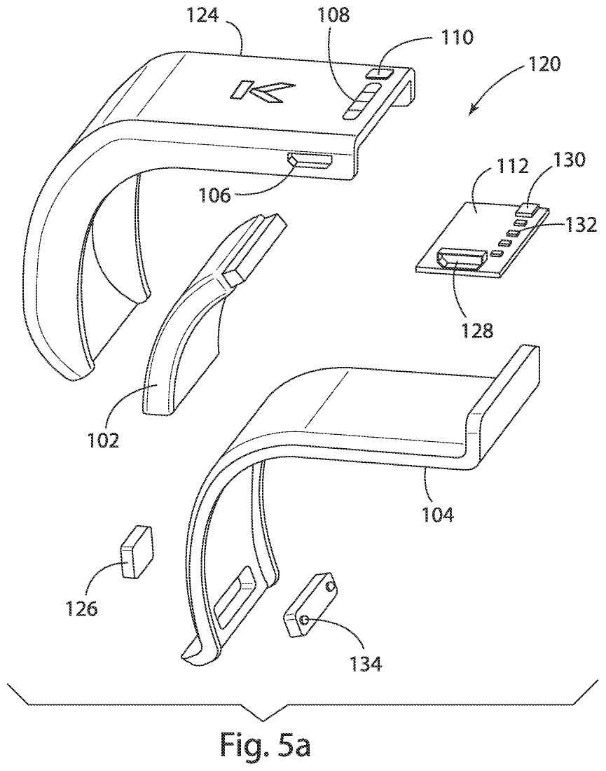

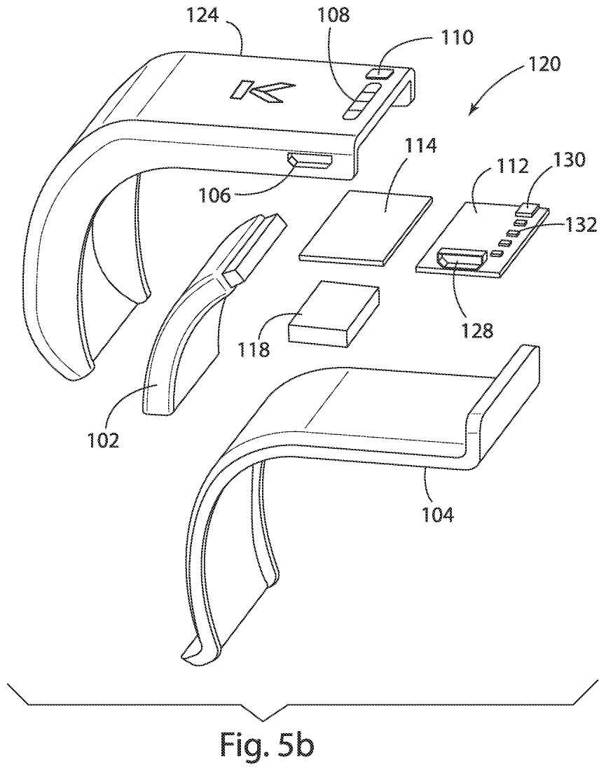

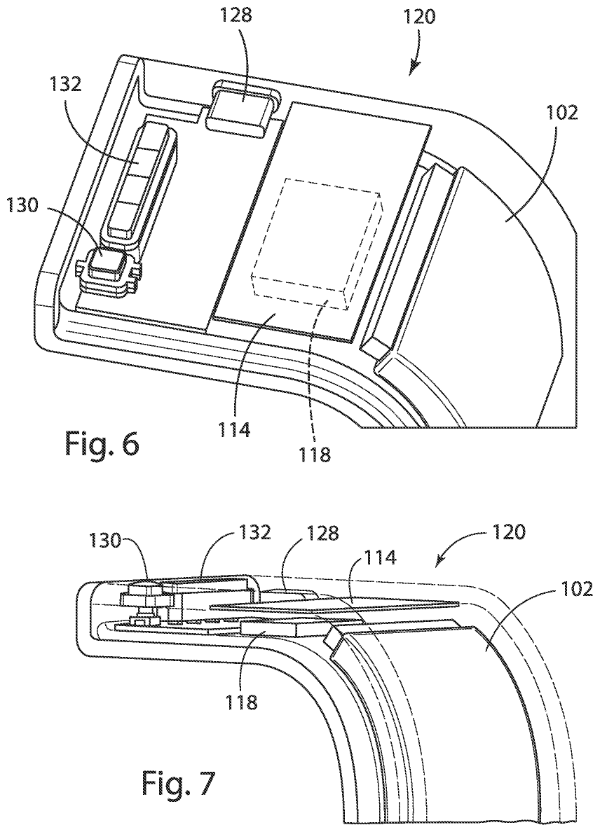

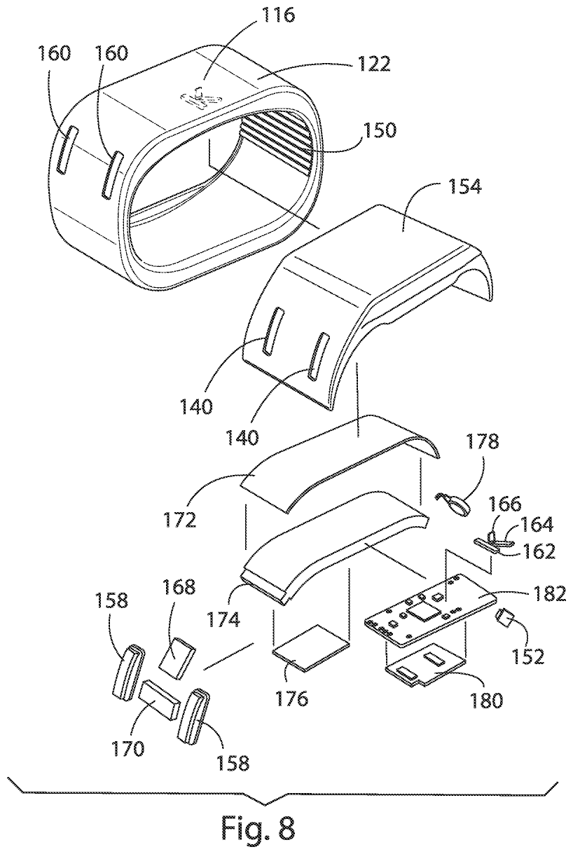

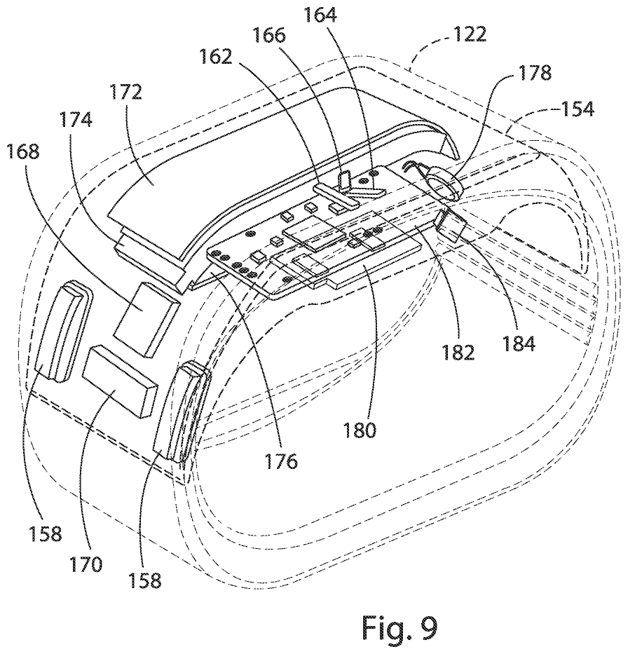

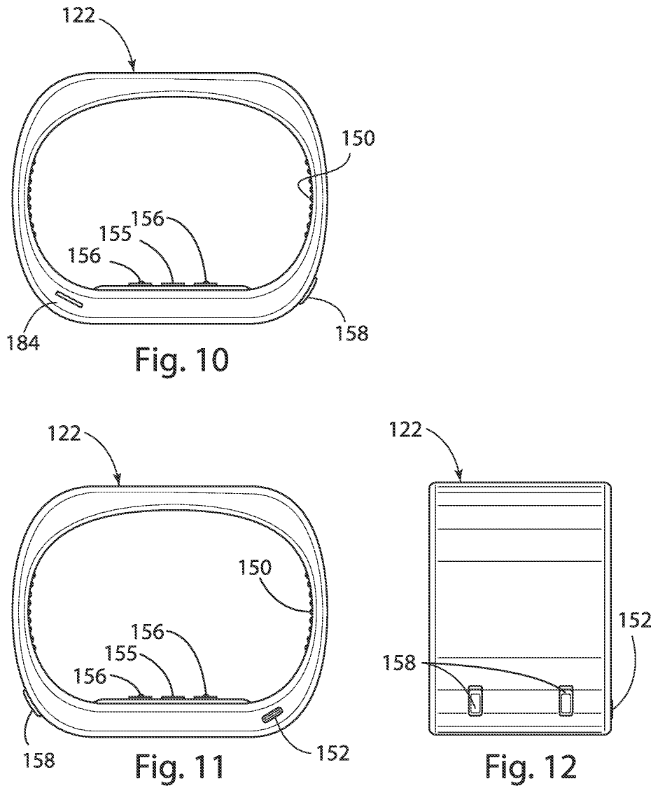

FIGS. 1-12 show one approach to the present embodiments. FIG. 1 illustrates an exemplary unassembled perspective wearable device shown as a wrist band and power bank according to one embodiment. FIG. 2 illustrates an assembled perspective wearable device and power bank of FIG. 1. FIG. 3 illustrates an exemplary unassembled perspective view of the wearable device and circuitry. FIG. 4 illustrates an exemplary assembled perspective wrist band and circuitry of FIG. 3. FIG. 5a illustrates an exemplary exploded view of a power bank with pogo pad charging. FIG. 5b illustrates and exemplary exploded view of a power bank with wireless charging. FIG. 6 illustrates an exemplary perspective cutaway view of a power bank with wireless charging capability of FIG. 5b. FIG. 7 illustrates an exemplary planar cutaway view of a power bank with wireless charging capability of FIG. 5b. FIG. 8 illustrates an exemplary exploded bottom perspective view of the wrist band. FIG. 9 illustrates an exemplary assembled bottom perspective view of the wrist band with the wristband material shown in hidden lines. FIG. 10 illustrates a front view of the exemplary wrist band of FIG. 1. FIG. 11 illustrates a back view of the exemplary wrist band of FIG. 1. And, FIG. 12 illustrates a side view of the exemplary wrist band of FIG. 1.

The figures show a monitor/power band assembly 100 with a wearable monitor 122 and battery bank 120. Power band 120 is shaped to the outer circumference/contours of monitor 122.

Power band 120 has a battery 102; a bottom enclosure 104; a body opening 106 for mini-usb port 128; power band body window or opening 108 to allow viewing of LED strip 132; opening 110 to allow switch 130 to extend therefrom; a printed circuit board 112; wired charger connection (e.g., Mini-USB port) 128 to wire charge power band 120; optional indicia 116 (FIG. 1). Power band body 124 (can be solid, opaque or transparent). Power band button 130 allows a user to switch on/off the battery charging and or to check the status of the battery power band LED strip 132 to show the battery state of charge.

In the embodiment of power band 124 with wireless charging, an RX coil 114 (and optionally with magnetic or metal strip) for optional wireless charging is provided. RX coil printed circuit board (PCB) (FIG. 5b) 118 for wireless charging embodiment is also provided.

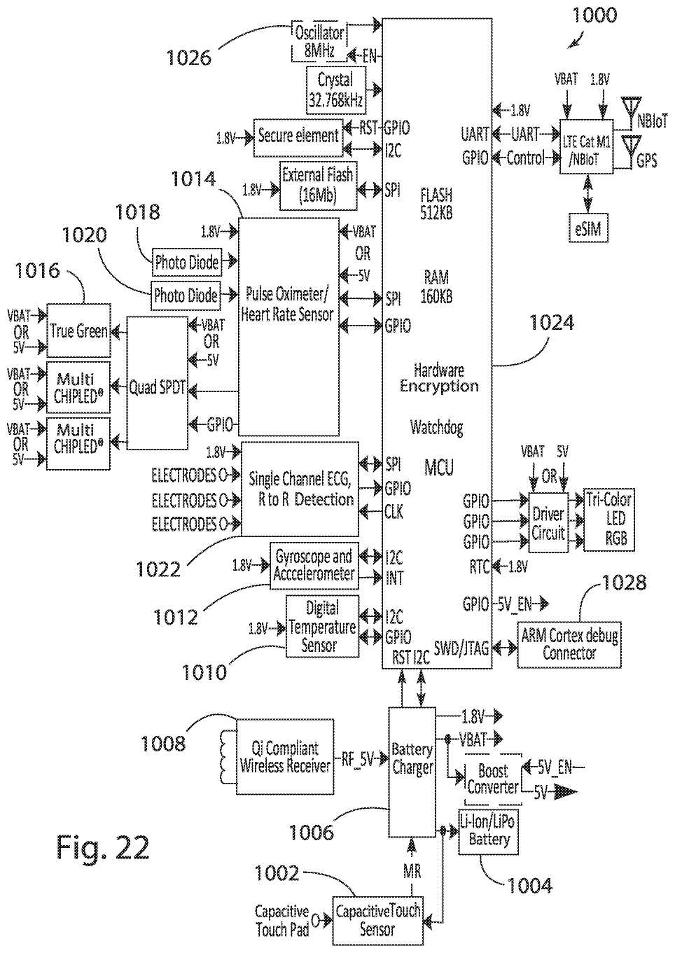

In the embodiment of power band 124 with direct charging with pogo pads, power band magnet 126 to hold pogo pins 134 and pogo pads 158 in wired charging embodiment (FIG. 5a). Wearable device can also have wrist band black glass 136 to keep the rest of the light from the rest of the band, ribbed and ribbed portion 150 of wrist band for air circulation switch 154 for wrist band; and a plastic encasement 154 to hold one or more components and circuit boards for monitor wearable device 122. Wearable device 122 may have a light source LED 155; with optical sensors 156 to read reflection from LED 155. The wearable device 122 as shown has apertures 160 for the pogo pads to extend through. As shown in FIG. 8, battery connection 168 is for attaching Charging Pack and magnet 170 is also for attaching Charging Pack. Referring to FIG. 8, a Bluetooth antenna 172, wearable monitor battery 174; shield 176 (sits under wearable monitor battery); vibrator/haptic 178; an LED and senor printed circuit board 180; a monitor printed circuit board 182 (see also, FIGS. 22-24); and a touch Pad 184 or NTC Sensor are found (See, FIGS. 10 and 9).

FIG. 13 illustrates an exemplary legend of messages from the exemplary LED signal 164, shown as a capital K. FIG. 13 shows a legend 200 for LEDs to show activities such as shown in listing 138 for indicia 116. As shown for exemplary purposes, three LEDs include blue indicia LED 162, green indicia LED 164, and 166 red indicia LED 166.

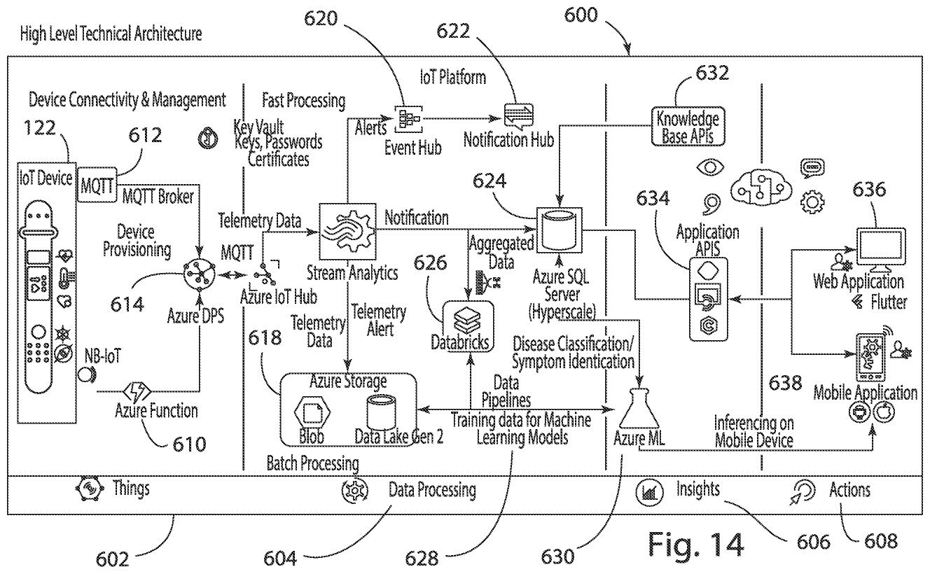

FIG. 14 illustrates a schematic for a system for use in implementing methods, techniques, devices, and applications of the present embodiments. FIG. 14 displays or represents the data flow from the IoT Device wearable device that monitors health-related symptom data of the wearer gathered by the wearable device, transfers the data to a cloud-based IoT Platform application where it is aggregated and processed. The processing of user symptom data consists of disease classification and the cloud-based application uses machine learning (ML) to train ML models for purposes of creating a user digital identity and of learning how a disease expresses itself in symptoms during the user's daily activities. FIG. 14 has labels included for the schematic. For example, the schematic shows the high level architecture IoT platform 600 dived into things 602, data processing 604, insights 606 and actions 608 having the wearable IoT device 122 as described herein with an MQTT 612 (a piece of software running on a computer (running on-premises or in the cloud), and could be self-built or hosted by a third party. The broker acts as a post office.) and a narrow band IoT Azure function 610 with and Azure DPS 614. Azure DPS is a helper service for managing provisioning IoT devices withe Azure IoT Hub at scale. It provides zero-touch, just-in-time provisioning of IoT devices with Azure IoT Hub in an automated fashion without the need for human interaction. Under data processing 604 there is batch processing 618; an event hub 620 leading to a notification hub. Data bricks 626 receive notifications and aggregate date 624 received notifications and knowledge based APIs 632 and application APIs. APIs are open source application programming interfaces you can access with the HTTP protocol. Also known as public APIs, they have defined API endpoints and request and response formats. Training data for machine learning models 628 output to Azure storage 618 and ML 630 as shown. Actions 698 are send to mobile applications 638 and/or the Web application 636.

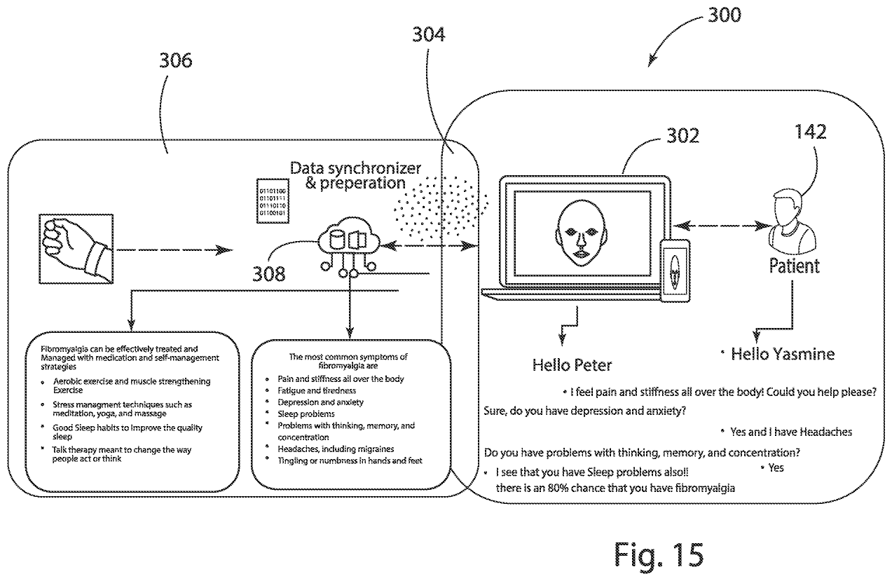

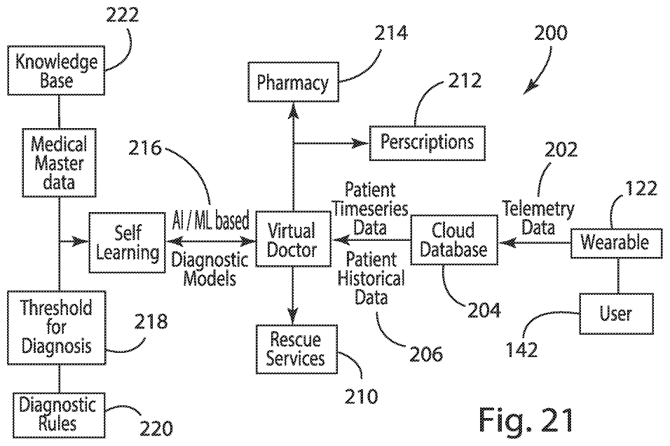

FIG. 15 illustrates an exemplary schematic representation of an application session with a user of the present embodiments. FIG. 15 displays or represents how the health-related symptom data of the wearer is transferred to a computer-based application that includes a chatbot. The user logs into this application and interacts with this chatbot by responding to a series of questions at 300, posed by the chatbot 302, or Virtual Doctor as displayed in FIG. 21, to the user 142 and related to the user symptom data and the context of this data, including the activities of the user associated with the symptom data. The responses of the user to the health- and wellness-related questions are used, together with the user symptom data acquired from the wearable device 306, by the cloud-based 304 application to determine and inform at 308 the cloud-based application users of possible conditions/diseases related to the patient's symptom data and the patient's responses to application chatbot, in a question-response session with the chatbot.

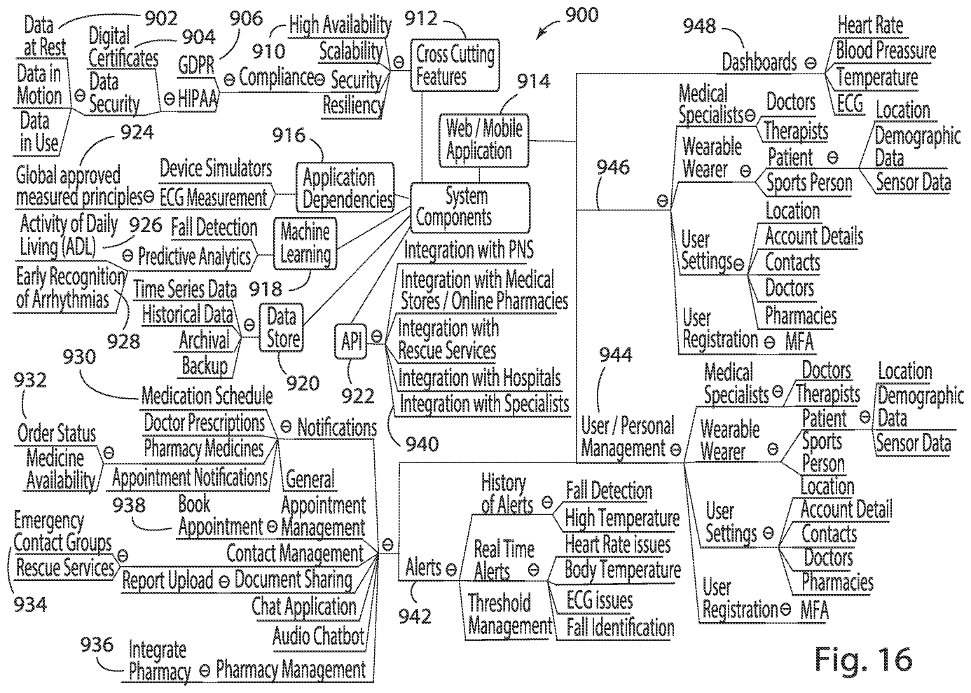

FIG. 16 illustrates a functional decomposition 900 of the present embodiments including the inputs and outputs. FIG. 16 displays or represents a functional decomposition of the system, including display of system inputs and system outputs. The inputs of the system of this invention include Data, at Rest and in Motion and in Use at 902. Inputs to the system also include Global Approved measured principles, Activity of Daily Life, and Early Recognition of Arrhythmias. Inputs to the system also include Order Status and Medicine Availability. Inputs also include Emergency Contact Groups and Rescue Services 934.

Outputs of the system include outputs to the Web/Mobile Application of the system, outputs to the wearable device/wristband system components, outputs to the APIs of the system and outputs to the Alerts of the system. The Web/Mobile Application of the system provide system functions, including Dashboards to Patient symptom data and to system users. System user functions include those function for Medical Specialists, Wristband Wearer, User Settings and User Registration. The Web/Mobile Application also provides function for User/Personal Management, including those for Medical Specialists, Wristband Wearer, User Settings and User Registration. The system provides outputs related to Alert functions, including History of Alerts, Real Time Alerts and Threshold Management. Other exemplary labeled items include order status 932, medication schedule 930; book appointment 938, integrate pharmacy 936, early diagnosis arrhythmias 928, ADL 926, global approved measure principles 924, digital certificates 904; General Data Protection Regulation, applied set of EU rules on data protection and privacy (GDPR) 906; high quality scalability 910; application dependencies 916, machine learning 918, cross cutting features 912, web/mobile application 914; data store 920; API 922, integrations 940; alerts 942, dashboards 948, settings, registration users and health professionals 946; and user and personal management 944.

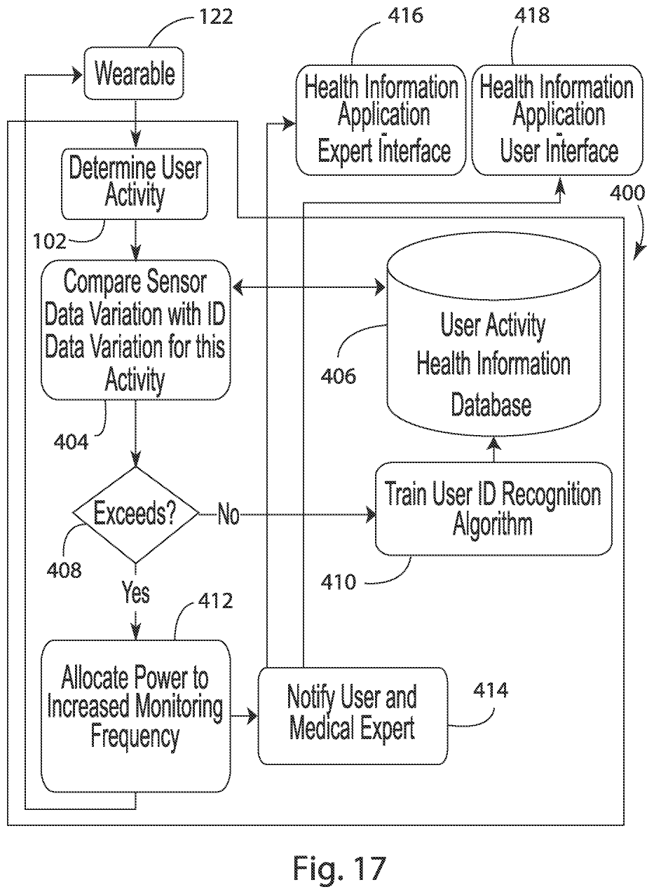

FIG. 17 illustrates an exemplary flow diagram of the present embodiments showing activity monitoring. FIG. 17 displays or represents the logic flow of the system as relates to the creation, maintenance and correlation of user symptom data with the daily activity of the user. The User Activity Health Information Database contains labels for user daily activity that are associated by the system with user symptom data using a learned User Digital Identifier (ID). This figure also displays how the association, between user symptom data and user daily activity, is reviewed by a Medical Expert. This figure also displays how the system manages the allocation of electrical Power and may alter the allocation of electrical power to realize Increased Monitoring Frequency by checking whether user symptom data acquired by the system from the user varies more than expected based on user symptom data history. As shown in the figure, flow diagram 400 receives input from the wearable device 122 to determine user activity 102 then comparing sensor data variation with ID data variation for this activity 404. If the data exceeds a variation at 408 the systems moves to the train user ID recognition algorithm at 410, which is outputted to the user activity health information database 406. The sensor data exceeds the data variation at 408, increased power monitoring frequency is implemented at 412 and can notify the user and health care professional at 414. The notify at step 414 proceeds to the expert interface 416 and user interface 418.

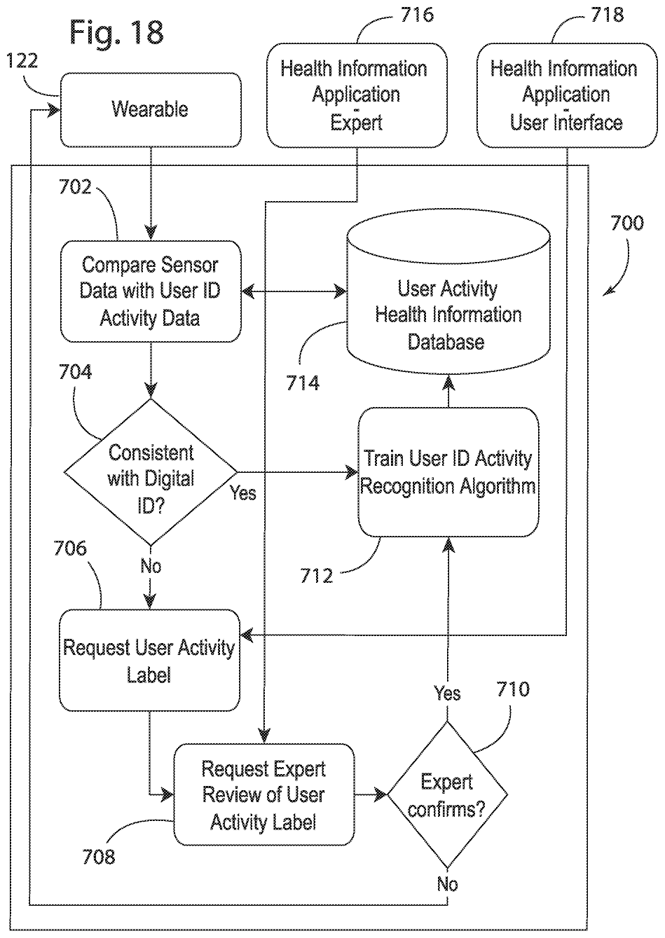

FIG. 18 illustrates an exemplary flow diagram of the present embodiments showing digital identification. FIG. 18 displays or represents the logic of the system as it relates to the creation, maintenance and use of a user Digital ID based on symptom sensor data acquired from the wearable wristband of the system. This figure also displays how its association of symptom sensor data, and the assessment of the rate of variation of user symptom sensor data, is correlated by the system with User Activity as displayed in FIG. 17. As shown in the Figure, device 122 data enters the flow diagram 700 where it is compared with user ID activity data at step 702. If the data are consistent within a predetermined threshold at 704, the date is outputted to train the user ID at 712, which is outputted to the user activity database 714. If the device data is not consistent with the digital ID at 704, a request for a user activity is generated at 706 from user interface 718 and an expert review of the label at 708. If the expert 716 confirms at 710, the activity is outputted to train the algorithm at 712.

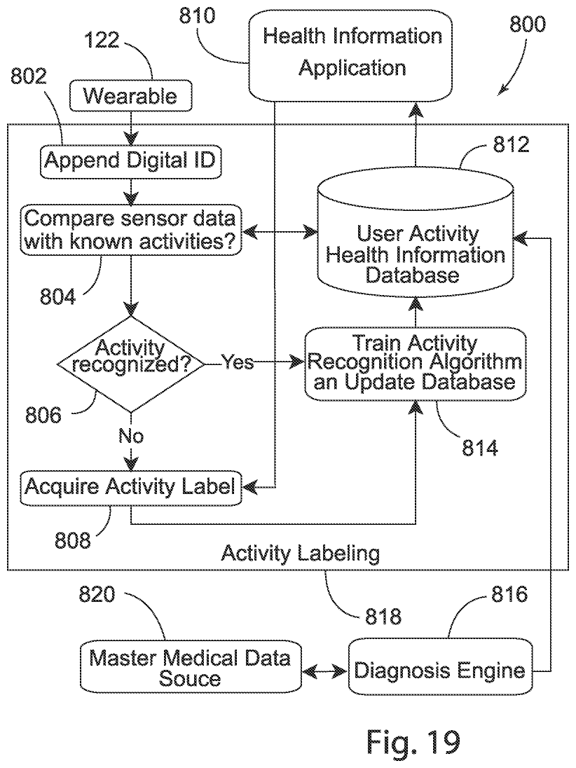

FIG. 19 illustrates an exemplary flow diagram of the present embodiments showing activity labeling. FIG. 19 displays or represents the logic flow of the system as it relates to the labeling user daily activity, including the training of the User Activity Recognition Algorithm. User symptom data acquired from the wearable, as displayed in FIG. 17, is appended to the User Digital ID of FIG. 18, then compared with user symptom data associated with the activity labels of the user's Digital ID in the User Activity Health Information Database. An activity is said to be recognized if the Recognition Algorithm recognizes and classifies the data acquired from the user wearable as characteristic of the user performing said activity. This figure also displays how the User Activity Health Information can access the Diagnosis Engine which intern exchanges information with a Master Medical Data Source. As shown in the Figure, algorithm 800 for activity labeling 818 receives data from wearable 122 which proceeds to append the digital ID at 802. This leads to a comparison of sensor data with known activities at 804. If it is recognized at 806, the algorithm proceeds to train at 814 and outputted to database 812 which can then be accessed by the application 810. Database 812 also receives data from a master medical data source 820 and diagnosis engine 816. If an activity from the wearable 122 is not recognized at 808, the application 810 will acquire an activity label at 808.

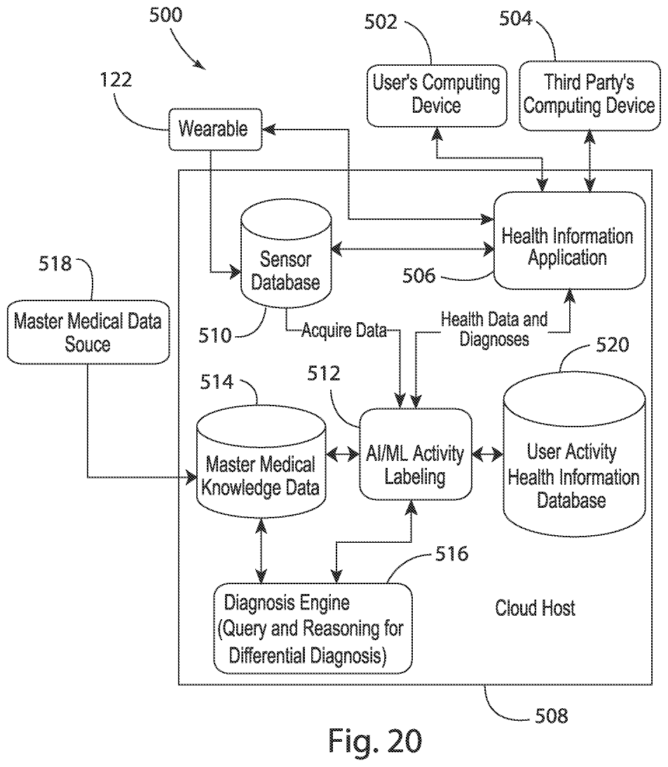

FIG. 20 illustrates an exemplary flow diagram of the present embodiments showing health data acquisition and proposed diagnoses using machine learning. FIG. 20 displays or represents the logic of the system as it relates to the recognition and classification and labeling of user symptom data as characteristic of a user daily activity. The system Labeling function makes use of an AI/ML-enabled Recognition Algorithm and the User Activity Information Database and is able to exchange information with the Master Medical Knowledge Data that accesses a Master Medical Data Source. The AI/ML Activity Labeling function may access the Diagnosis Engine which in turn may exchange data with the Master Medical Knowledge Data of the system. As shown in algorithm 500 to cloud host 508, the wearable 122 provides data to a sensor database 510 which is provided to the AI/ML activity labeling algorithm, which also inputs master medical data 514 from a master medical data source 518 and from application 506 and user activity database 520. The processes work with the diagnosis engine as shown. The system application 506 can output data to the user 502 and third party 504 such as a health care professional.

FIG. 21 illustrates an exemplary flow diagram of the present embodiments showing a virtual doctor application. FIG. 21 displays or represents the flow of information from the Patient or User through the wearable, to the Virtual Doctor via the Cloud Database. This figure also displays or represents the AI/ML-based exchange of information, related to the Diagnostic Models, between the Virtual Doctor and Self Learning. This figure also displays or represents Self Learning access in formation from the Knowledge Base through the Medical Master data and accesses the Diagnostic Rules through the Threshold for Diagnosis component of the system. As shown in the figure, the virtual doctor can receive diagnostic models that are AI/ML generated at 216 from self learning from a knowledge base 222 from medical master data and diagnostic rules 220 for thresholds 218. The user 142 input from wearable device 122 is send at 202 to a cloud database 204 to the virtual doctor. A virtual doctor can output rescue services and the like at 210, pharmacy 214 for prescription 212.

FIGS. 22-24 show exemplary circuit boards to be placed in plastic encasement 154 which are labeled and are communicatively connected. Some of the components are described below.

FIG. 22 illustrates an exemplary circuitry configuration of the present embodiments for a wearable device. As shown, circuit board 1000 has a printed circuit board 1024, a cortex debug connector 1028, a battery 1004, a capacitive touch sensor 1002; a battery charger 1006, a wireless receiver 1008, a digital temperature sensor 1010, a gyroscope and accelerometer 1012; a single channel ECG 1022; an LED array 1016; photodiodes 1018 and 1020; a pulse oximeter/heart rate sensor 1014; and an oscillator 1026.

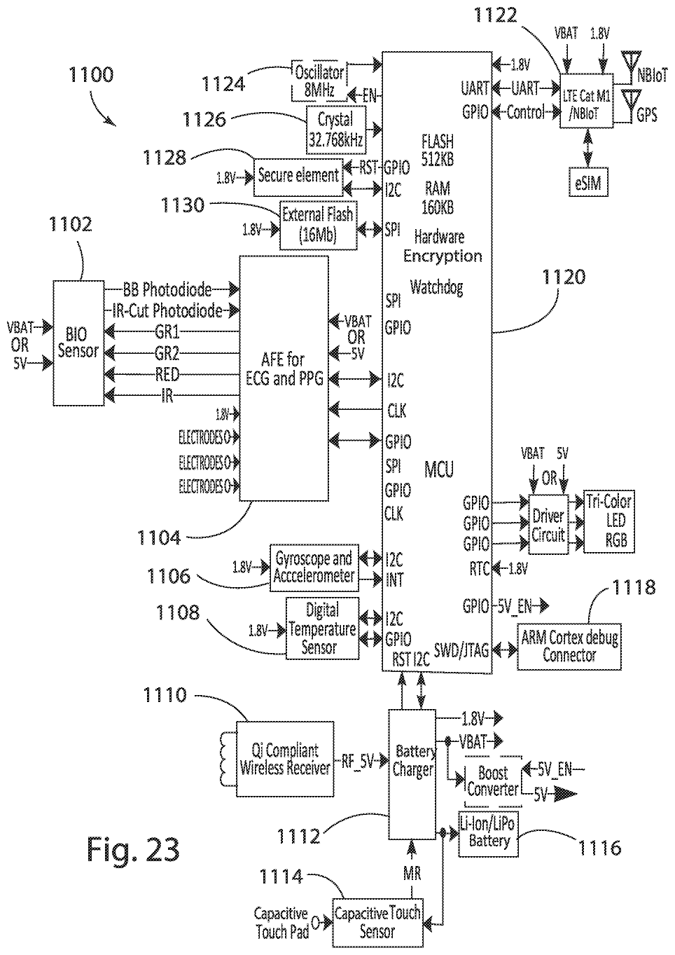

FIG. 23 illustrates an exemplary circuitry configuration of the present embodiments for a wearable device. As shown, circuit board 1100 has a printed circuit board 1120; and oscillator 1124; a crystal 1126; a secure element 1128; an external flash 1130; a biosensor 1102; an AFE for ECG and PPG 1104; a gyroscope and accelerometer 1106; digital temperature sensor 1108; a wireless receiver 1110; a battery charger 1112; a capacitive touch sensor 1114; a battery 1116; and an ARM Cortex debug connector 1118.

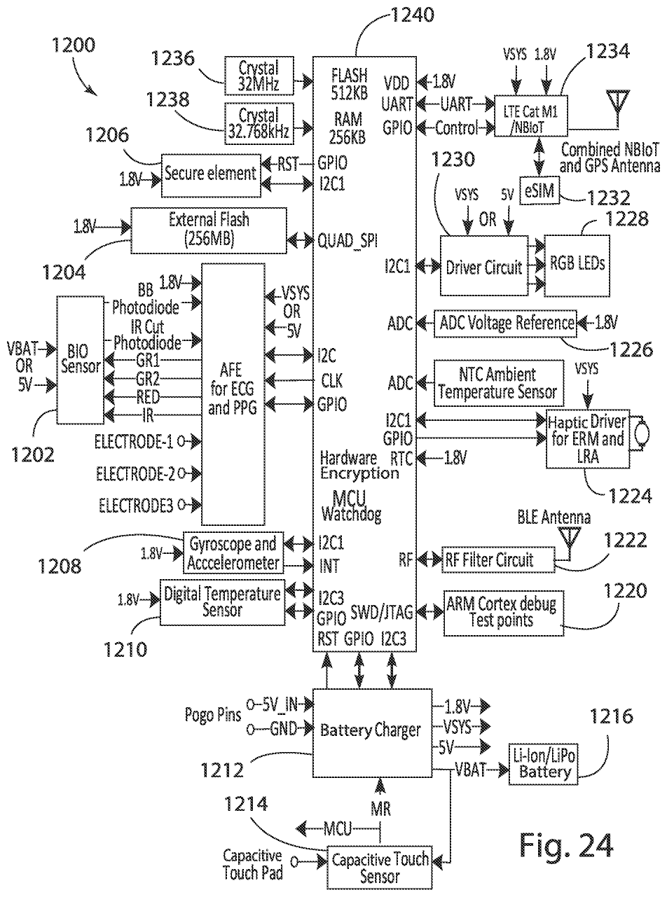

FIG. 24 illustrates an exemplary circuitry configuration of the present embodiments for a wearable device. As shown, circuit board 1200 has a printed circuit board 1240; a crystal 1236 and 1238; a secure element 1206; an external flash 1204; biosensor 1202; gyroscope and accelerometer 1208; digital temperature sensor 1210; battery charger 1212; capacitive touch sensor 1214; battery 1216; ARM cortex debug test points 1220; RF filter circuit 1222; haptic driver and LRA 1224; ADC voltage reference 1226; LEDs 1228; LED driver circuit 1230; eSIM 1232; and LTE Cat M1/NBIoT 1234.

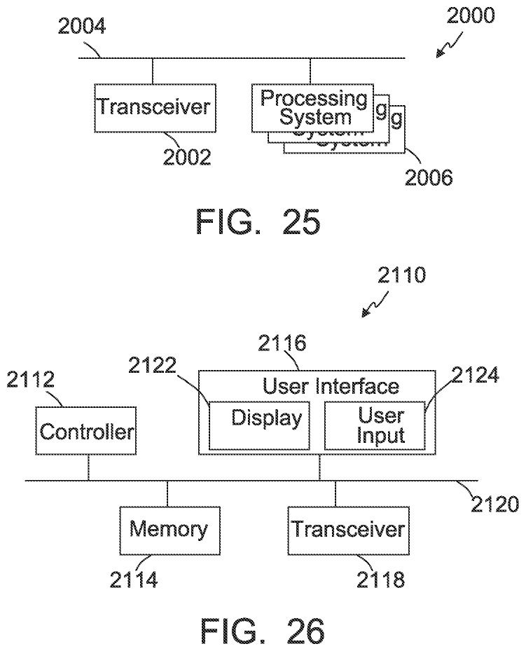

FIG. 25 illustrates a system that may be used in processing signals in accordance with at least some embodiments. In FIG. 25, there is illustrated a system 2000 that may be used in processing data in accordance with at least some embodiments. The system 2000 can include a received and/or transceiver 2002, one or more communication links, paths, buses or the like 2004, and one or more processing systems, chips or units 2006. The transceiver 2002 can be configured to receive the signal to be processed. The processing systems 2006 can be substantially any circuitry, circuits, chips, ASICs and/or combinations thereof that can implement the processing, which can include but is not limited to one or more of perform the segmenting, the transform series expansion, the calculations, summations, sampling, transmitting, storing, analyzing, reconstructing, synthesizing, transmitting and the like. Similarly, the processing system 2006 may include one or more processors, microprocessors, central processing units, logic, local digital storage, firmware and/or other control hardware and/or software. As such, the system may include multiple processing systems 2006 to implement the multiple cycles.

The methods, techniques, systems, devices, services, and the like described herein may be utilized, implemented and/or run on many different types of devices and/or systems. FIG. 26 illustrates a system for use in implementing methods, techniques, devices, apparatuses, systems, modules, units and the like in providing user interactive virtual environments in accordance with some embodiments. Referring to FIG. 26, there is illustrated a system 2110 that may be used for any such implementations, in accordance with some embodiments. One or more components of the system 2110 may be used for implementing any system, apparatus, module, unit or device mentioned above or below, or parts of such systems, apparatuses, modules, unit or devices, such as for example any of the above or below mentioned circuitry, chips, ASICs, systems, processing systems 1006, processors, and the like. However, the use of the system 2110 or any portion thereof is certainly not required.

By way of example, the system 2110 may comprise a controller or processor module 2112, memory 2114, one or more communication links, paths, buses or the like 2120, and in some instances a user interface 2116. A power source or supply (not shown) is included or coupled with the system 1100. The controller 2112 can be implemented through one or more processors, microprocessors, central processing unit, logic, local digital storage, firmware and/or other control hardware and/or software, and may be used to execute or assist in executing the steps of the methods and techniques described herein, and control various transforms, analysis, transmissions, storage, reconstruction, synthesis, windowing, measuring, communications, programs, interfaces, etc. The user interface 2116, when present, can allow a user to interact with the system 2100 and receive information through the system. In some instances, the user interface 2116 may includes a display 2122, LEDs, audio output, and/or one or more user inputs 2124, such as keyboard, mouse, track ball, touch pad, touch screen, buttons, track ball, etc., which can be part of or wired or wirelessly coupled with the system 1100.

Typically, the system 2100 further includes one or more communication interfaces, ports, transceivers 2118 and the like allowing the system 2100 to at least receive signals, which can be communicated wired or wirelessly over substantially any communication medium (e.g., over a distributed network, a local network, the Internet, communication link 2120, other networks or communication channels with other devices and/or other such communications). Further the transceiver 2118 can be configured for wired, wireless, optical, fiber optical cable or other such communication configurations or combinations of such communications.

The system 2100 comprises an example of a control and/or processor-based system with the controller 2112. Again, the controller 2112 can be implemented through one or more processors, controllers, central processing units, logic, software and the like. Further, in some implementations the controller 2112 may provide multiprocessor functionality.

The memory 2114, which can be accessed by the controller 2112, typically includes one or more processor readable and/or computer readable media accessed by at least the controller 2112, and can include volatile and/or nonvolatile media, such as RAM, ROM, EEPROM, flash memory and/or other memory technology. Further, the memory 2114 is shown as internal to the system 2110; however, the memory 2114 can be internal, external or a combination of internal and external memory. The external memory can be substantially any relevant memory such as, but not limited to, one or more of flash memory secure digital (SD) card, universal serial bus (USB) stick or drive, other memory cards, hard drive and other such memory or combinations of such memory. The memory 2114 can store code, software, executables, scripts, data, signals, samples, coefficients, programming, programs, media stream, media files, identifiers, log or history data, user information and the like.

One or more of the embodiments, methods, processes, approaches, and/or techniques described above or below may be implemented in one or more processor and/or computer programs executable by a processor-based system. By way of example, such a processor based system may comprise the processor based system 2100, a computer, an encoder, an analog-to-digital converter, a player device, etc. Such a computer program may be used for executing various steps and/or features of the above or below described methods, processes and/or techniques. That is, the computer program may be adapted to cause or configure a processor-based system to execute and achieve the functions described above or below. For example, such computer programs may be used for implementing any embodiment of the above or below described steps, processes or techniques. As another example, such computer programs may be used for implementing any type of tool or similar utility that uses any one or more of the above or below described embodiments, methods, processes, approaches, and/or techniques. In some embodiments, program code modules, loops, subroutines, etc., within the computer program may be used for executing various steps and/or features of the above or below described methods, processes and/or techniques. In some embodiments, the computer program may be stored or embodied on a computer readable storage or recording medium or media, such as any of the computer readable storage or recording medium or media described herein.

Accordingly, some embodiments provide a processor or computer program product comprising a medium configured to embody a computer program for input to a processor or computer and a computer program embodied in the medium configured to cause the processor or computer to perform or execute steps comprising any one or more of the steps involved in any one or more of the embodiments, methods, processes, approaches, and/or techniques described herein. For example, some embodiments provide one or more computer-readable storage mediums storing one or more computer programs for use with a computer simulation, the one or more computer programs configured to cause a computer and/or processor based system to execute steps.

Many of the functional units described in this specification have been labeled as systems, modules, units, etc., in order to more particularly emphasize their implementation independence. For example, a system or module may be implemented as a hardware circuit comprising custom VLSI circuits or gate arrays, off-the-shelf semiconductors such as logic chips, transistors, or other discrete components. A system and/or module may also be implemented in programmable hardware devices such as field programmable gate arrays, programmable array logic, programmable logic devices or the like.

Some or all of the systems and/or modules may also be implemented in software for execution by various types of processors. An identified system and/or module of executable code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a system or module of executable code could be a single instruction, or many instructions, and may even be distributed over several different code segments, among different programs, and across several memory devices. Similarly, operational data may be identified and illustrated herein within systems or modules, and may be embodied in any suitable form and organized within any) suitable type of data structure. The operational data may be collected as a single data set, or may be distributed over different locations including over different storage devices, and may exist, at least partially, merely as electronic signals on a system or network.

While the invention herein disclosed has been described by means of specific embodiments, examples and applications thereof, numerous modifications and variations could be made thereto by those skilled in the art without departing from the scope of the invention set forth in the claims.

### Definitions

1. Wearable: (technology) is any kind of electronic device designed to be worn on the user's body that includes electronic sensors and is able to transmit the data gathered by its sensors, wirelessly or in a wired fashion, to a computer programming application.

2. Sensor Data: is acquired from a user's wearable when the wearable device has been registered to the computer programming application, for example, the user's: Heart rate, Blood oxygen level (Pulse Oximeter), Skin Temperature, Echocardiogram (ECG), _Ambient Temperature in the user's environment, User movement (Accelerometer and Gyroscope and Vibration), User location (GPS).

3. Activities of Daily Life (ADL): Activities that an average person engages in during their daily life such as running, resting, exercise, work, recreation, eating and sleeping.

4. Labeling of ADL: The assignment of a word or phrase to a set of data acquired by a wearable such as the data acquired at an instant in time or over a period of time. Labeling provides contextual data for algorithm optimization.

5. Differential Diagnosis: A differential diagnosis is a list of possible conditions, with each condition's probability or likelihood, each whose description in terms of symptoms is consistent with the sensor data acquired from the user's wearable device and the user's responses to questions posed by the Conversational AI Chatbot.

6. Virtual Doctor Application: A computer programming application hosted in the Cloud or on a computing device, mobile or stationary, that can be connected to a computer network with Internet access, exchanges information with wearable devices and performs healthcare functions such as:
Wearable Device Registration and Management
User Registration and Management
Assignment of digital ID to wearable devices and users
Visualization of data or Dashboard, such as visual rendering of sensor data acquired by the wearable device and historical sensor data acquired by the wearable device
Labeling of ADL
Wearable communications, such as exchange of information, such as sensor data acquired from the wearable device and notifications to the user
Mobile to Cloud communications
Voice- or text-based Conversational AI chatbot that:
Accesses Master Medical Knowledge databases, locally or over the Internet
Compares sensor data acquired from the user's wearable device with data symptom data in the Medical Knowledge Base
Computes and displays differential diagnoses from sensor date acquired from a wearable device and presents, in text and verbally this differential diagnoses
Suggests corrective steps
Image capture using camera
Diagnose diseases
Staying healthy
Patient appointment workflow
Order and received medication notifications
Input from Third Party
System designed to accept inputs from third parties (e.g., doctor, clinic, etc.)

7. Transmitted Data to the Cloud: Data acquired from a user's wearable device, acquired from sensors of the wearable device, and transmitted over a network by the wearable to the Health Information Application, whether hosted in the Cloud or on another networked computer.

8. Sensor Data Storage: Sensor data is acquired from the user's wearable and, once transmitted, stored in computer storage accessible by the Health Information Application.

9. User Login Credentials: The username and password, or other form of authentication, used by a user to log into the Health Information Application.

10. Health Information Application: While logged into the Health Information Application the user can: input data; request reports and histories; input responses to questions posed to the user by the application; choose a ‘label’ from a selection of Activities of Daily Life (ADL) such as for example: Walking; Cycling; Running; Idle; Sleeping; 200+ additional activities.

11. Master Medical Knowledge database: Any source of medical knowledge that consists of medical condition names together with the symptoms associated with each condition name, where symptoms consist of a physical or mental features which are regarded as indicating a condition of disease, particularly such a feature that is apparent to the patient.

12. Digital Identity (Digital ID): A digital identity is information used by computer systems to represent an external agent, such as a person, organization, application, or device.

13. User Activity Healthcare Information: The User Activity Healthcare Information refers to the linking of ADL labels to sensor data acquired by the Healthcare Information Application from a user's wearable device. This information links, for example: User's digital ID; ADL labels; Sensor data acquired from a user's wearable device.

14. Diagnosis Engine: The Diagnosis Engine refers to a computer programming application that: Accesses a user's sensor data using the user's Digital ID; Queries the Master Medical Knowledge Database using the user's sensor data to form such queries, reasons about the results of such queries, and outputs a Differential Diagnosis.

1.-6. (canceled)

7. A charger to charge a wearable device while in use, the charger configured to transfer energy to the wearable device by one of wirelessly or direct connection through complementary pogo pads, the charger comprising: an LED strip to indicate power level; magnets to attach to the wrist band during charging; a plastic polycarbonate enclosure; a battery in the power pack having a capacity of >400 mAh.

8. A charger to charge a wearable device while in use, the charger configured to transfer energy to the wearable device wirelessly, the charger comprising: an LED strip to indicate power level; magnets to attach to the wrist band during charging; a plastic polycarbonate enclosure; a battery in the power pack having a capacity of >400 mAh, wherein the charger has a wireless charging mechanism and a Metallic (ferrous) Strip on its top side to hold it against a magnet in wireless charger.

9. The charger of claim 7, wherein the charger uses a direct electrical connection using pogo pads, the pogo pads configured to be received by enlarged pogo pads on the wearable device whereby contact is maintained as a user moves and flexes the band or to adjust to the users variation in wrist size.

10. The charger of claim 9, wherein a power band magnet is placed close to the pogo pads and configured to be adjacent to a metal pad or complimentary magnet on the wearable device.

11.-15. (canceled)
